(19) United States
(12) Patent Application Publication
(10) Pub. No.: US 2024/0023887 A1
(43) Pub. Date: Jan. 25, 2024

(54) ULTRASOUND PUPIL CHANGE-BASED ANESTHESIA DEPTH MONITORING SYSTEM AND DETECTION METHOD

(71) Applicant: SUZHOU SCIENCE & TECHNOLOGY TOWN HOSPITAL, Suzhou (CN)

(72) Inventors: Chen WANG, Suzhou (CN); Yanjun DENG, Suzhou (CN)

(21) Appl. No.: 18/251,508

(22) PCT Filed: May 9, 2022
(86) PCT No.: PCT/CN2022/091607

§ 371 Date: May 2, 2023

(30) Foreign Application Priority Data
Jul. 20, 2021 (CN) 202110822186.9

(51) Int. Cl. A61B 5/00; A61B 8/10; A61B 3/11

(52) U.S. Cl. CPC A61B 5/4821; A61B 8/10; A61B 3/112; A61B 5/746

(57) ABSTRACT

The present disclosure discloses an ultrasound pupil change-based anesthesia depth monitoring system, including: an ultrasound monitoring apparatus, which includes a head-mounted mounting member and two miniature ultrasonic probes, the two miniature ultrasonic probes being symmetrically arranged at both sides of the head-mounted mounting member, and the miniature ultrasonic probes abutting against the face and obtaining ultrasonic pupil images by means of ultrasonic waves; an ultrasound host, which includes a display, the ultrasonic pupil images obtained by the miniature ultrasonic probes being transmitted to the ultrasound host, and the ultrasound host processing the ultrasonic pupil images and displaying pupil images, real-time change values of diameters of pupils, and the like by means of the display; and a shading patch, which is applied to the eyelids. The present disclosure further discloses a detection method of an ultrasound pupil change-based anesthesia depth monitoring system. Compared with the prior art, the present disclosure has the advantages: A patient wears a head-mounted ultrasound monitoring apparatus, the miniature probes of the ultrasound monitoring apparatus monitor pupil changes while the patient is in an anesthesia/sedation state, thereby realizing real-time dynamic monitoring of an anesthesia depth.

### TECHNICAL FIELD

The present disclosure belongs to the technical field of monitoring of an anesthesia depth, in particular to an ultrasound pupil change-based anesthesia depth monitoring system and a detection method.

### BACKGROUND

Anesthesia can be described as states of analgesia, unconsciousness and muscle paralysis induced by specific drugs. Any overdose or under medication during anesthesia will bring additional risks. In the past, anesthesiologists determined an analgesia depth according to experiences, and evaluated the inhibition of nociceptive pathways by using physiological reactions, such as exercises, tachycardia, tears or sweating. These symptoms occurred relatively late, insensitively and inaccurately. A processed encephalogram (EEG) is widely used to monitor drug-induced hypnosis. However, in terms of analgesia and unconsciousness, current monitoring methods cannot fully meet clinical needs.

Pupil monitoring is an important indicator in a traditional anesthesia observation process. Regular and quantitative monitoring of pupils can reflect physiological awakening, reflect and evaluate autonomic nerve activities, reflect heart rate changes, and achieve non-contact and non-invasive measurement of a heart rate variability, is helpful to make a judgment on conditions of patients suffering from coma, convulsion, shock, poisoning, respiratory failure and circulatory failure, especially for patients suffering from craniocerebral injury. An intracranial injury part can be determined.

Pupil changes can reflect an anesthesia depth, but existing pupil diameter measurement methods include doctor's manual measurement, an infrared pupilometer or a hand-held ultrasonic pupilometer, by which, a patient needs to cooperate with a doctor to do some poses, and measurement requirements are relatively high. As a result, pupillary responses cannot be quantitatively measured, and the accuracy of observing pupils cannot be guaranteed.

### SUMMARY

The present disclosure aims to provide an ultrasound pupil change-based anesthesia depth monitoring system and a detection method. A patient wears a head-mounted ultrasound monitoring apparatus. When the patient is in an anesthesia state, miniature ultrasonic probes of the ultrasound monitoring apparatus obtain ultrasound images of the pupils of the eyes of the patient, and pupil changes are displayed by means of a display of an ultrasound host, which realizes monitoring of an anesthesia depth.

In order to achieve the above objectives, in one aspect, the present disclosure adopts an ultrasound pupil change-based anesthesia depth monitoring system, including:
an ultrasound monitoring apparatus, which comprises a head-mounted mounting member and two miniature ultrasonic probes, wherein the two miniature ultrasonic probes are symmetrically arranged at both sides of the head-mounted mounting member; one miniature ultrasonic probe corresponds to one eye; and the miniature ultrasonic probes abut against the face and obtain ultrasonic pupil images by means of ultrasonic waves;
an ultrasound host, which includes a display, wherein the ultrasonic pupil images obtained by the miniature ultrasonic probes are transmitted to the ultrasound host; and the ultrasound host processes the ultrasonic pupil images and displays pupil images, real-time change values of diameters of the pupils, an average value of the diameters of the pupils of the two eyes, a pupil diameter change trend chart, and other data information by means of the display; and
a shading patch, which is applied to the eyelids, wherein the shading patch eliminates impact of indoor light on pupillary light reflex, and locates the ultrasound monitoring apparatus.

As a further description of the above-mentioned technical solution:
the two miniature ultrasonic probes are separately arranged at outer sides of the two eyes or at lower eyelids of the two eyes.

As a further description of the above-mentioned technical solution:
the head-mounted mounting member includes a first head-mounted mounting member, a second head-mounted mounting member and a mask type mounting member.

As a further description of the above-mentioned technical solution:
the first head-mounted mounting member includes a cambered first elastic headband;
the two miniature ultrasonic probes are symmetrically arranged at two ends of the first elastic headband; and the two miniature ultrasonic probes are separately arranged at the outer sides of the two eyes.

As a further description of the above-mentioned technical solution:
the second head-mounted mounting member comprises a second elastic headband and a nose pad; the nose pad is stuck on the nose bridge of the user's face; the two miniature ultrasonic probes are symmetrically arranged at two sides of the nose pad; and the miniature ultrasonic probes abut against the lower eyelids of the eyes.

As a further description of the above-mentioned technical solution:
the mask type mounting member includes a mask and ties; the mask includes a first mask body and a second mask body; the first mask body is connected with a breathing line;
the second mask body abuts against the face contour around the eyes; the two miniature ultrasonic probes are arranged in mounting slots of the second mask body; the miniature ultrasonic probes abut against the lower eyelids of the eyes; and the ties are fixedly mounted on the mask.

As a further description of the above-mentioned technical solution:
the shading patch is a medical cold compress eye patch.

As a further description of the above-mentioned technical solution:
the ultrasound host further includes a sound warning module or a light warning module.

In another aspect, the present disclosure adopts a detection method of an ultrasound pupil change-based anesthesia depth monitoring system, including the following steps:
1) making a patient wear the ultrasound monitoring apparatus, wherein the two miniature ultrasonic probes separately correspond to the left and right eyes, and the miniature ultrasonic probes are adjusted to abut against the face;
2) applying the shading patch to the eyelids of the patient, wherein the shading patch locates the ultrasound monitoring apparatus;
3) transmitting and receiving, by the miniature ultrasonic probes, ultrasonic waves to monitor the left and right eyes, and obtaining ultrasonic pupil images; and
4) transmitting the ultrasonic pupil images obtained by the miniature ultrasonic probes to the ultrasound host, processing, by the ultrasound host, the ultrasonic pupil images, and displaying pupil images, real-time change values of diameters of the pupils, an average value of the diameters of the pupils of the two eyes, a pupil diameter change trend chart, and other data information by means of the display.

As a further description of the above-mentioned technical solution:
in the step 4), transmitting manners of the ultrasonic pupil images include Bluetooth, wireless and wired.

In summary, the above-mentioned technical solution is used, so that the present disclosure has the beneficial effects as follows.

1. In the present disclosure, before anesthesia, a patient first wears the head-mounted ultrasound monitoring apparatus. The miniature ultrasonic probes of the ultrasound monitoring apparatus abut against the face, but they will not compress the eyes. In an anesthesia state, the two miniature ultrasonic probes of the ultrasound monitoring device obtain the ultrasonic pupil images of the two eyes of the patient. The ultrasound host processes the ultrasonic pupil images, and displays pupil images, real-time values of diameters of the pupils, an average value of the diameters of the pupils of the two eyes, a pupil diameter change trend chart, and other data information by means of the display. An anesthesia depth is judged by pupil changes, and anesthesia depth monitoring is realized. The pupil images can be switched to observe one eye or both eyes at the same time, and a pupillary distance can be marked.

2. In the present disclosure, the two miniature ultrasonic probes can be arranged at the outer sides of the two eyes or at the lower eyelids of the two eyes. The positions of the miniature ultrasonic probes are flexible, which reduces service restrictions. The positions of the probes can be set flexibly according to requirements, to ensure an effect of anesthesia depth monitoring. Correspondingly, different head-mounted mounting members can be used to mount the miniature ultrasonic probes.

### BRIEF DESCRIPTION OF THE DRAWINGS

In order to explain the technical solutions of the embodiments of the present disclosure more clearly, the following will briefly introduce the accompanying drawings used in the embodiments. It should be understood that the drawings in the following description only illustrate some embodiments of the present disclosure and thus shall not be deemed as limiting the scope. Those of ordinary skill in the art can obtain other related drawings based on these drawings without creative work.

FIG. 1 is a flow diagram of a detection method of an ultrasound pupil change-based anesthesia depth monitoring system.

FIG. 2 is a schematic structural diagram I of a first head-mounted mounting member in an ultrasound pupil change-based anesthesia depth monitoring system.

FIG. 3 is a schematic structural diagram II of a first head-mounted mounting member in an ultrasound pupil change-based anesthesia depth monitoring system.

FIG. 4 is a schematic structural diagram II of a first elastic headband in an ultrasound pupil change-based anesthesia depth monitoring system.

FIG. 5 is a schematic structural diagram I of a second head-mounted mounting member in an ultrasound pupil change-based anesthesia depth monitoring system.

FIG. 6 is a schematic structural diagram II of a second head-mounted mounting member in an ultrasound pupil change-based anesthesia depth monitoring system.

FIG. 7 is a schematic structural diagram of a mask type mounting member in an ultrasound pupil change-based anesthesia depth monitoring system.

### ILLUSTRATIONS IN THE DRAWINGS

1: miniature ultrasonic probe; 2: first elastic headband; 3: second elastic headband; 4: nose pad; 5: mask; 51: first mask body; 52: second mask body; and 6: tie.

### DETAILED DESCRIPTION OF THE EMBODIMENTS

The technical solutions in the embodiments of the present disclosure will be described clearly and completely below in combination with the accompanying drawings of the embodiments of the present disclosure. Apparently, the described embodiments are only part of the embodiments of the present disclosure, not all embodiments. Based on embodiments of the present disclosure, all other embodiments obtained by those of ordinary skill in the art without creative work shall fall within the scope of protection of the present disclosure.

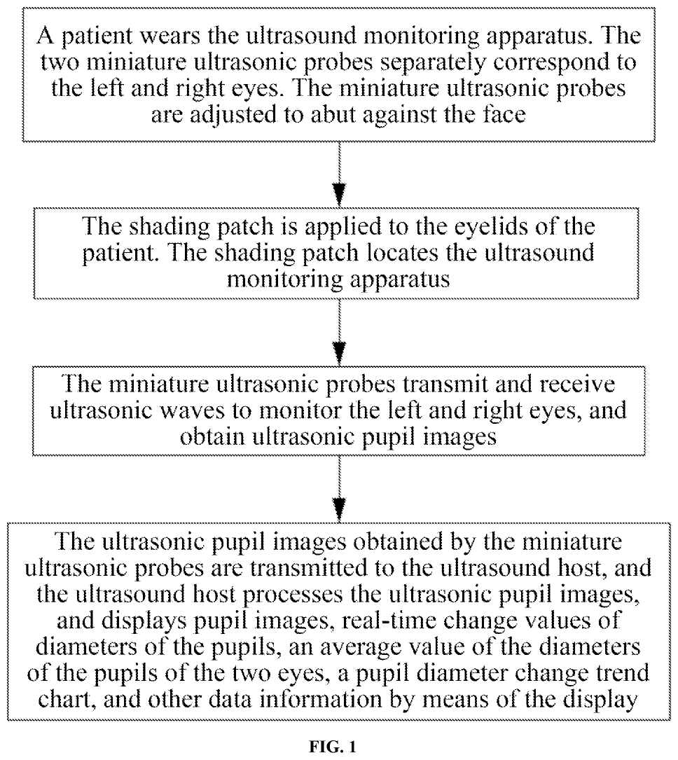

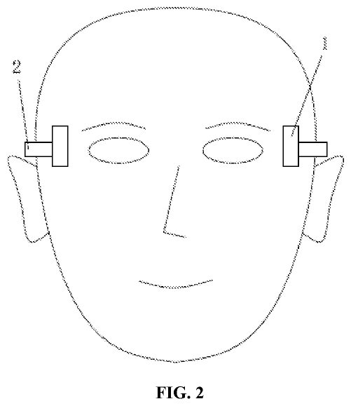

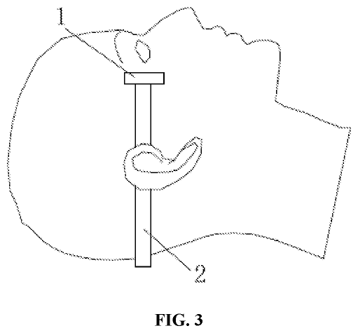

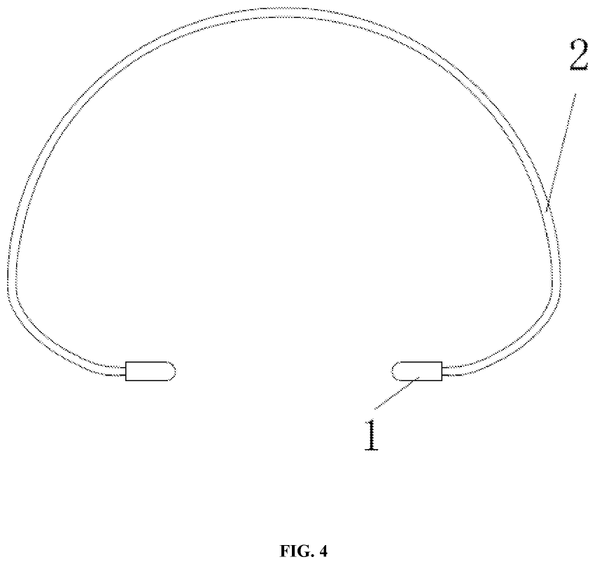

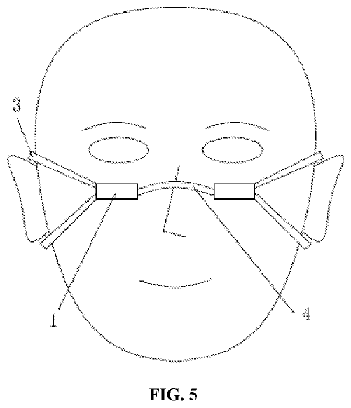

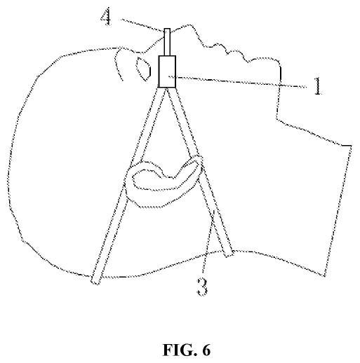

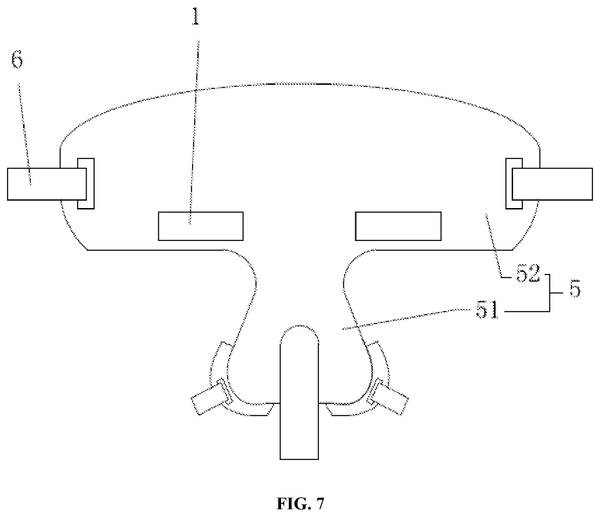

Referring to FIG. 1 to FIG. 7, the present disclosure provides a technical solution: In one aspect, the present disclosure adopts an ultrasound pupil change-based anesthesia depth monitoring system, including:
an ultrasound monitoring apparatus, which includes a head-mounted mounting member and two miniature ultrasonic probes 1, wherein the two miniature ultrasonic probes 1 are symmetrically arranged at both sides of the head-mounted mounting member; one miniature ultrasonic probe 1 corresponds to one eye; and the miniature ultrasonic probes 1 abut against the face and obtain ultrasonic pupil images by means of ultrasonic waves;
an ultrasound host, which includes a display, wherein the ultrasonic pupil images obtained by the miniature ultrasonic probes 1 are transmitted to the ultrasound host; and the ultrasound host processes the ultrasonic pupil images and displays pupil images, real-time change values of diameters of the pupils, an average value of the diameters of the pupils of the two eyes, a pupil diameter change trend chart, and other data information by means of the display; and
a shading patch, which is applied to the eyelids, wherein the shading patch eliminates impact of indoor light on pupillary light reflex, and locates the ultrasound monitoring apparatus.

The two miniature ultrasonic probes 1 are separately arranged at outer sides of the two eyes or at lower eyelids of the two eyes. The positions of the miniature ultrasonic probes 1 are flexibly set according to a requirement, to better monitor pupil changes, thereby realizing monitoring of an anesthesia depth.

The head-mounted mounting member includes a first head-mounted mounting member, a second head-mounted mounting member and a mask type mounting member.

The first head-mounted mounting member includes a cambered first elastic headband 2. The two miniature ultrasonic probes 1 are symmetrically arranged at two ends of the first elastic headband 2. The two miniature ultrasonic probes 1 are separately arranged at the outer sides of the two eyes. The ultrasound monitoring apparatus is convenient to wear.

The second head-mounted mounting member includes a second elastic headband 3 and a nose pad 4. The nose pad 4 is stuck on the nose bridge of the user's face. The two miniature ultrasonic probes 1 are symmetrically arranged at two sides of the nose pad 4. The miniature ultrasonic probes 1 abut against the lower eyelids of the eyes. When the miniature ultrasonic probes 1 are arranged at the lower eyelids, coupling agents are arranged between the miniature ultrasonic probes 1 and the skin. On the one hand, the miniature ultrasonic probes 1 fit the face, and on the other hand, angles of the ultrasonic probes can be adjusted by means of adjusting thicknesses of the coupling agents.

The mask type mounting member includes a mask 5 and ties 6. The mask 5 includes a first mask body 51 and a second mask body 52. The first mask body 51 is connected with a breathing line. The second mask body 52 abuts against the face contour around the eyes. The two miniature ultrasonic probes 1 are arranged in mounting slots of the second mask body 52. The miniature ultrasonic probes 1 abut against the lower eyelids of the eyes. The ties 6 are fixedly mounted on the mask 5. The mask 5 is fixed by the ties 6. The ties are arranged on both the first mask body 51 and the second mask body 52, to ensure a fixing effect. Compared with a traditional breathing mask, the mask has improved in its shape. The second mask body 52 that matches the face contour is added. The second mask body 52 has a space inside for placing the miniature ultrasonic probes. By the arrangement of the second mask body 52, compared with the traditional breathing mask, the mask will not compress the eyes and the probes during fixing, and will not leak air, to ensure a pupil change monitoring effect.

The shading patch is a medical cold compress eye patch, which can prevent the eyes from being dry and astringent if a user is in an anesthesia state for a long time, and can also prevent the impact of indoor light on pupillary light reflex.

The ultrasound host also includes a sound warning module or a light warning module. The ultrasound host sets a baseline value and a warning value for a pupil change. When the pupil change exceeds the warning value, the ultrasound host will make a sound or light warning.

In another aspect, the present disclosure adopts a detection method of an ultrasound pupil change-based anesthesia depth monitoring system, including the following steps:
1) A patient wears the ultrasound monitoring apparatus. The two miniature ultrasonic probes 1 separately correspond to the left and right eyes. The miniature ultrasonic probes 1 are adjusted to abut against the face.
2) The shading patch is applied to the eyelids of the patient. The shading patch locates the ultrasound monitoring apparatus.
3) The miniature ultrasonic probes 1 transmit and receive ultrasonic waves to monitor the left and right eyes, and obtain ultrasonic pupil images.
4) The ultrasonic pupil images obtained by the miniature ultrasonic probes 1 are transmitted to the ultrasound host, and the ultrasound host processes the ultrasonic pupil images, and displays pupil images, real-time change values of diameters of the pupils, an average value of the diameters of the pupils of the two eyes, a pupil diameter change trend chart, and other data information by means of the display.

In the step 4), transmitting manners of the ultrasonic pupil images comprise Bluetooth, wireless and wired. Data transmission can be performed in various manners.

Working principle: Before anesthesia, a patient first wears the head-mounted ultrasound monitoring apparatus. The miniature ultrasonic probes of the ultrasound monitoring apparatus abut against the face, but they will not compress the eyes. In an anesthesia state, the two miniature ultrasonic probes of the ultrasound monitoring device obtain the ultrasonic pupil images of the two eyes of the patient. The ultrasound host processes the ultrasonic pupil images, and displays pupil images, real-time values of diameters of the pupils, an average value of the diameters of the pupils of the two eyes, a pupil diameter change trend chart, and other data information by means of the display. An anesthesia depth is judged by pupil changes, and anesthesia depth monitoring is realized. The pupil images can be switched to observe one eye or both eyes at the same time, and a pupillary distance can be marked. The two miniature ultrasonic probes can be arranged at the outer sides of the two eyes or at the lower eyelids of the two eyes. The positions of the miniature ultrasonic probes are flexible, which reduces service restrictions. The positions of the probes can be set flexibly according to requirements, to ensure an effect of anesthesia depth monitoring. Correspondingly, different head-mounted mounting members can be used to mount the miniature ultrasonic probes.

The above descriptions are only specific preferred implementation modes of the present disclosure, but the scope of protection of the present disclosure is not limited thereto. For any person skilled in the art, within the technical scope disclosed by the present disclosure, equivalent substitutions or changes made according to the technical solution of the present disclosure and an inventive idea of the present disclosure shall all fall within the scope of protection of the present disclosure.

What is claimed is:

1. An ultrasound pupil change-based anesthesia depth monitoring system, comprising:
an ultrasound monitoring apparatus, which comprises a head-mounted mounting member and two miniature ultrasonic probes (1), wherein the two miniature ultrasonic probes (1) are symmetrically arranged at both sides of the head-mounted mounting member; one miniature ultrasonic probe (1) corresponds to one eye; and the miniature ultrasonic probes (1) abut against the face and obtain ultrasonic pupil images by means of ultrasonic waves;
an ultrasound host, which comprises a display, wherein the ultrasonic pupil images obtained by the miniature ultrasonic probes (1) are transmitted to the ultrasound host; and the ultrasound host processes the ultrasonic pupil images and displays pupil images, real-time change values of diameters of the pupils, an average value of the diameters of the pupils of the two eyes, a pupil diameter change trend chart, and other data information by means of the display; and
a shading patch, which is applied to the eyelids, wherein the shading patch eliminates impact of indoor light on pupillary light reflex, and locates the ultrasound monitoring apparatus.

2. The ultrasound pupil change-based anesthesia depth monitoring system according to claim 1, wherein the two miniature ultrasonic probes (1) are separately arranged at outer sides of the two eyes or at lower eyelids of the two eyes.

3. The ultrasound pupil change-based anesthesia depth monitoring system according to claim 2, wherein the head-mounted mounting member comprises a first head-mounted mounting member, a second head-mounted mounting member and a mask type mounting member.

4. The ultrasound pupil change-based anesthesia depth monitoring system according to claim 3, wherein the first head-mounted mounting member comprises a cambered first elastic headband (2); the two miniature ultrasonic probes (1) are symmetrically arranged at two ends of the first elastic headband (2); and the two miniature ultrasonic probes (1) are separately arranged at the outer sides of the two eyes.

5. The ultrasound pupil change-based anesthesia depth monitoring system according to claim 3, wherein the second head-mounted mounting member comprises a second elastic headband (3) and a nose pad (4); the nose pad (4) is stuck on the nose bridge of the user's face; the two miniature ultrasonic probes (1) are symmetrically arranged at two sides of the nose pad (4); and the miniature ultrasonic probes (1) abut against the lower eyelids of the eyes.

6. The ultrasound pupil change-based anesthesia depth monitoring system according to claim 3, wherein the mask type mounting member comprises a mask (5) and ties (6); the mask (5) comprises a first mask body (51) and a second mask body (52); the first mask body (51) is connected with a breathing line; the second mask body (52) abuts against the face contour around the eyes; the two miniature ultrasonic probes (1) are arranged in mounting slots of the second mask body (52); the miniature ultrasonic probes (1) abut against the lower eyelids of the eyes; and the ties (6) are fixedly mounted on the mask (5).

7. The ultrasound pupil change-based anesthesia depth monitoring system according to claim 1, wherein the shading patch is a medical cold compress eye patch.

8. The ultrasound pupil change-based anesthesia depth monitoring system according to claim 1, wherein the ultrasound host further comprises a sound warning module or a light warning module.

9. A detection method of an ultrasound pupil change-based anesthesia depth monitoring system, comprising the following steps:
1) making a patient wear the ultrasound monitoring apparatus, wherein the two miniature ultrasonic probes (1) separately correspond to the left and right eyes, and the miniature ultrasonic probes (1) are adjusted to abut against the face;
2) applying the shading patch to the eyelids of the patient, wherein the shading patch locates the ultrasound monitoring apparatus;
3) transmitting and receiving, by the miniature ultrasonic probes (1), ultrasonic waves to monitor the left and right eyes, and obtaining ultrasonic pupil images; and
4) transmitting the ultrasonic pupil images obtained by the miniature ultrasonic probes (1) to the ultrasound host, processing, by the ultrasound host, the ultrasonic pupil images, and displaying pupil images, real-time change values of diameters of the pupils, an average value of the diameters of the pupils of the two eyes, a pupil diameter change trend chart, and other data information by means of the display.

10. The detection method of the ultrasound pupil change-based anesthesia depth monitoring system according to claim 9, wherein in the step 4), transmitting manners of the ultrasonic pupil images comprise Bluetooth, wireless and wired.
